(19) United States
(12) Patent Application Publication
(10) Pub. No.: US 2010/0145266 A1
(43) Pub. Date: Jun. 10, 2010

(54) METHOD FOR LOADING STRUCTURED SURFACES

(76) Inventor: Michael Orlowski, Bonn (DE)

Correspondence Address: MEYERTONS, HOOD, KIVLIN, KOWERT & GOETZEL, P.C., P.O. BOX 398, AUSTIN, TX, 78767-0398, US

(21) Appl. No.: 12/524,135

(22) PCT Filed: Jan. 21, 2008
(86) PCT No.: PCT/DE08/00095

§ 371 Date: Jan. 19, 2010

Related U.S. Application Data
(60) Provisional application No. 60/899,637, filed on Feb. 6, 2007.

(30) Foreign Application Priority Data
Jan. 22, 2007 (DE) 10 2007 003 184.1

(51) Int. Cl. A61M 29/00; A61F 2/82; A61L 33/00

(52) U.S. Cl. 604/96.01; 623/1.36; 427/2.25

(57) ABSTRACT

The invention relates to a method for loading structured surfaces that are preferably made of a polymer material with the pharmacological agent paclitaxel as well as the coated medical products obtained by means of said method.

### BACKGROUND OF THE INVENTION

1. Field of the Invention

The present invention relates to a method for loading textured surfaces, preferably such surfaces which are composed of a polymeric material, with the pharmacological agent paclitaxel; and to the coated medical devices obtained by means of said method.

2. Description of the Relevant Art

Nowadays, implantation of vessel grafts such as stents has become a well-established surgical intervention for the treatment of stenoses. In this context, so-called restenosis (recurrent stenosis), i.e. the reocclusion of the vessel is a frequently occurring complication. There's no exact definition of the term restenosis to be found in literature. The most frequently used morphological definition of restenosis defines restenosis as a reduction of the vessel diameter to less than 50% of the normal value subsequent to successful PTA (percutaneous transluminal angioplasty). Said definition describes an empirically determined value and its hemodynamic meaning and association with clinical symptoms lack scientific background. In practice, clinical deterioration in a patient is often considered a sign for the occurrence of restenosis in the previously treated vessel section.

Restenosis following stent implantation is one of the major causes for further hospitalization. Vessel traumas induced during stent implantation cause inflammatory reactions which play a decisive role in the healing process during the first seven days. In the recent past, it has also been found that stents provided with a drug-eluting coating may cause late thromboses, i.e. in addition to restenosis the stent may also lead to long-term problems such as late thromboses.

To avoid such problems, a so-called “biological stenting” may be performed using only a coated catheter balloon without any stent, i.e. the vessels are dilated at a constricted site by the dilatation of a coated catheter balloon, wherein, while the catheter balloon is dilated for a short period of time, a sufficient amount of pharmacological agent is transferred to the vessel wall to avoid re-constriction or reocclusion of the vessel due to the dilatation of the vessel and the delivery of active agents.

Such coated catheter balloons are already known from WO 2005/089855 A1 and the international patent application WO 2004/028582 A1 discloses multifold balloons which are coated, especially within the folds, with a composition of a pharmacological agent and a contrast medium. A method for spray coating catheter balloons is described in WO 2004/006976 A1.

### SUMMARY OF THE INVENTION

Due to the fact that the active agent paclitaxel has proven to be particularly useful in the prevention of restenosis, as can be seen especially in European patent no. EP 0 706 376 B1, while coated stents, however, are disadvantageous with respect to the late thromboses described above, it is an objective to apply the active agent paclitaxel onto a catheter balloon in such manner that a coating is created which is easily detached from the balloon and can be effectively transferred to the vessel wall.

Said objective is resolved by the technical teaching of the independent claims. Further advantageous embodiments of the invention result from the dependent claims, the description and the examples.

It has been found that a coating method of the following type is especially suited for resolving said objective.

Said method for loading or coating dilatable catheter balloons includes:
I) providing a dilatable catheter balloon;
II) providing a solution of paclitaxel in dimethyl sulfoxide;
III) texturing the surface of the dilatable catheter balloon;
IV) wetting the surface of the dilatable catheter balloon with the solution of paclitaxel in dimethyl sulfoxide;
V) applying a solvent which is capable of precipitating paclitaxel onto the surface of the dilatable catheter balloon wetted with the solution of paclitaxel in dimethyl sulfoxide;
VI) drying the wetted surface of the dilatable catheter balloon;

In another embodiment, multifold balloons are coated when in expanded state by a method that includes:
I) providing a dilatable catheter balloon;
IIa) providing a solution of paclitaxel in dimethyl sulfoxide;
IIb) expansion of the balloon that includes folds so that inner surfaces of the folds become accessible;
III) texturing the surface of the dilatable, expanded catheter balloon;
IV) wetting the surface of the dilatable catheter balloon with the solution of paclitaxel in dimethyl sulfoxide;
V) applying a solvent which is capable of precipitating paclitaxel onto the surface of the dilatable catheter balloon wetted with the solution of paclitaxel in dimethyl sulfoxide;
VI) drying the wetted surface of the dilatable catheter balloon;
VII) refolding of the foldable balloon back into its compressed state.

The method for loading or coating catheter balloons and multifold balloons is carried out proceeding from step I) to VI) or respectively I) to VII), wherein it is evident that the order regarding steps I) and II) can be interchanged.

Moreover, steps IV) to VI) may be repeated several times. Step V) that includes the application of a solvent capable of precipitating paclitaxel has to be performed at least once, but does not necessarily have to be repeated in a second coating process. Thus, it is also possible to perform steps IV) to VI) i.e. steps I) to VI) in the first coating process and to include only steps IV) to VI) when the coating process is repeated.

### BRIEF DESCRIPTION OF THE DRAWINGS

Advantages of the present invention will become apparent to those skilled in the art with the benefit of the following detailed description of embodiments and upon reference to the accompanying drawings in which:

FIGS. 1A-D depict coronary angiography of a patient before and after implantation with a stent;

FIGS. 2A-D depict coronary angiography of another patient before and after implantation of a stent in the LAD coronary artery;

FIGS. 3A-B depict coronary angiography of the patient of FIGS. 2A-D before and after implantation of a stent in the LCX coronary artery

FIGS. 4A-B depict coronary angiography of the patient of FIGS. 2A-D before and after implantation of a stent in the Right Coronary Artery

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

Surprisingly it has been found that a coating method of the following type is especially suited for resolving said objective.

Said method for loading or coating dilatable catheter balloons includes:
I) providing a dilatable catheter balloon;
II) providing a solution of paclitaxel in dimethyl sulfoxide;
III) texturing the surface of the dilatable catheter balloon;
IV) wetting the surface of the dilatable catheter balloon with the solution of paclitaxel in dimethyl sulfoxide;
V) applying a solvent which is capable of precipitating paclitaxel onto the surface of the dilatable catheter balloon wetted with the solution of paclitaxel in dimethyl sulfoxide;
VI) drying the wetted surface of the dilatable catheter balloon;

Any commercially available dilatable catheter balloon may be used as catheter balloon. Preferably, so called multifold balloons are used, as described for example in the international patent application WO 94/23787 A1 by David H. Rammler, Labintelligence, USA; or the international patent application WO 03/059430 A1 by Scimed Life Sciences, Inc., USA; or the international patent application WO 2004/028582 A1 by Prof. Dr. Ulrich Speck or the European Patent No. EP 0519063 B1 by Medtronic Inc., USA.

Such balloons are provided with folds or wings forming essentially closed cavities when the balloon is in its compressed state but bending outward during dilatation and being capable of releasing substances contained in the folds or respectively of pressing said substances against the vessel wall.

Such balloons are advantageous since the substances enclosed in the folds or respectively paclitaxel enclosed in the folds are protected from being detached too soon during the insertion of the catheter.

To protect the active agent paclitaxel from early detachment from the catheter balloon, paclitaxel may also be incorporated or embedded into a carrier substance, preferably a polymeric carrier. In order to apply said carrier or respectively polymeric carrier containing paclitaxel onto the catheter balloon, the carrier substance is added to the solution of DMSO and paclitaxel. Such DMSO solutions containing paclitaxel and the carrier substance are then applied onto the catheter balloon using conventional methods, in particular spattering or dipping methods. Suitable carriers are such substances which are also used as balloon material, in particular polymeric and polymerizable substances as listed further below.

Also in such cases where the coating, i.e. the paclitaxel, is not protected by the folds of a multifold balloon or where the paclitaxel is not incorporated into a carrier, a sufficient amount of the pure active agent paclitaxel may be applied onto the catheter balloon so that even including a calculated amount of paclitaxel in the range of about 30% of the total amount being detached prematurely during the insertion of the catheter balloon, there is still a sufficiently high and therapeutically active amount of paclitaxel present on the balloon once it has reached its target, which amount can then be transferred to the vessel wall during dilatation.

Thus, it is preferred to protect the active agent paclitaxel from premature detachment e.g. by application underneath the folds or by embedment into a carrier on the surface of the catheter balloon, although such a protection is not necessarily required.

In one embodiment, multifold balloons are coated when in expanded state so that a slightly modified method is used for coating multifold balloons, which method includes:
I) providing a dilatable catheter balloon;
IIa) providing a solution of paclitaxel in dimethyl sulfoxide;
IIb) expansion of the balloon that includes folds so that inner surfaces of the folds become accessible;
III) texturing the surface of the dilatable, expanded catheter balloon;
IV) wetting the surface of the dilatable catheter balloon with the solution of paclitaxel in dimethyl sulfoxide;
V) applying a solvent which is capable of precipitating paclitaxel onto the surface of the dilatable catheter balloon wetted with the solution of paclitaxel in dimethyl sulfoxide;
VI) drying the wetted surface of the dilatable catheter balloon;
VII) refolding of the foldable balloon back into its compressed state.

The method for loading or coating catheter balloons and multifold balloons is carried out proceeding from step I) to VI) or respectively I) to VII), wherein it is evident that the order regarding steps I) and II) can be interchanged.

Moreover, steps IV) to VI) can be repeated several times in the inventive coating methods. Step V) that includes the application of a solvent capable of precipitating paclitaxel has to be performed at least once, but does not necessarily have to be repeated in a second coating process. Thus, it is also possible to perform steps IV) to VI) i.e. steps I) to VI) in the first coating process and to include only steps IV) to VI) when the coating process is repeated.

Usually, the coating procedure is repeated one or two or three times, but said repetition is not obligatory. Even one coating procedure may be sufficient for the application of the required amount of paclitaxel onto the catheter balloon.

Generally, an amount of 0.5 μg to 50 μg of paclitaxel per mm² of the surface of the balloon catheter to be coated and preferably an amount of 1 μg to 20 μg of paclitaxel per mm² of the surface of the balloon catheter to be coated is applied onto the surface of the balloon catheter. Preferably, 10 to 1000 μg of paclitaxel are applied per catheter balloon and most preferably 20μ to 400 μg are applied onto the balloon per catheter balloon.

Paclitaxel is commercially available from several suppliers. Paclitaxel is known under the trademark name of Taxol® and is also designated with various synonymous names such as:

BMS 181339-01, BMS-181339, BMS-181339-01, Capxol, DRG-0190, DTS-301, Ebetaxel, Genaxol, Genexol, Genexol-PM, HSDB 6839, Intaxel, KBio2_{—}002509, KBio2_{—}005077, KBio2_{—}007645, KBio3_{—}002987, KBioGR_{—}002509, KBioSS_{—}002517, LipoPac, MBT 0206, MPI-5018, Nanotaxel, NCl60_{—}000601, Nova-12005, NSC 125973, NSC-125973, NSC125973, Onxol, Pacligel, Paxceed, Paxene, Paxoral, Plaxicel, QW 8184, SDP-013, TA1, Tax-11-en-9-on, TaxAlbin, Taxol A, Xorane or Yewtaxan.

Its chemical structure is as follows:

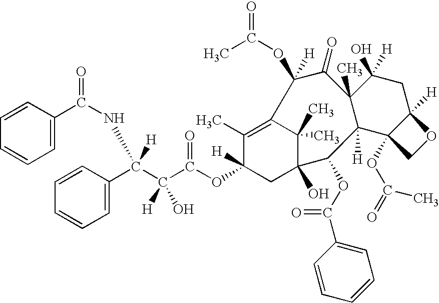

IUPAC nomenclature is as follows: [2aR-[2a,4,4a,6,9(R*,S*),11,12,12a,12b]]-(benzoylamino)-hydroxybenzene propionic acid 6,12b-bis-(acetyloxy)-12-(benzoyloxy)-2a-3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester).

Paclitaxel is highly soluble in dimethyl sulfoxide (DMSO) and methanol as well as in anhydrous ethanol, but is comparatively poorly soluble in water. Paclitaxel is especially stable at a pH between 3 and 5 and can be stored for long periods, whereas it is comparatively instable at alkaline pH.

Dimethyl sulfoxide (DMSO) is used as a solvent for paclitaxel. DSMO dissolves the required amounts of paclitaxel while corroding the material of the balloon catheter only to a small extent. Materials used for the balloon catheter are such materials as listed further below, wherein the following polymers are particularly preferred: polyamides, block copolymers of polyamide, polyether and polyester, polyurethanes, polyesters and polyolefins.

Furthermore, by successively increasing its water content DMSO can slowly reduce the solubility of paclitaxel in DMSO to a point where paclitaxel starts to precipitate or to crystallize. The structure of the precipitated paclitaxel is not clear but it has been shown that a paclitaxel coating obtained by the embodiments disclosed herein detaches particularly well from the catheter balloon and can be particularly well transferred to the vessel wall.

In the dimethyl sulfoxide paclitaxel is soluble in amounts of up to 150 mg/ml. Preferably 1 mg-150 mg, more preferably 10 mg to 90 mg and most preferably 40 mg to 60 mg of paclitaxel are dissolved in one ml of DMSO.

Moreover, DMSO is preferably used in dry form, i.e. in essentially anhydrous form. The water content of DMSO should not exceed 5 percent by volume, preferably 3 percent by volume and more preferably 1 percent by volume.

As already mentioned before, a carrier for the active agent paclitaxel may be added to the DMSO solution. Preferably, the carrier is a biostable or biodegradable polymer, preferably selected from the group of polymers disclosed further below as materials for the catheter balloon. The carrier substance(s) used may include a weight percentage of up to 70% by weight, preferably up to 50% by weight, more preferably up to 30% per weight with respect to the total weight of the solution.

According to the inventive methods, the surface of the catheter balloon is textured mechanically, chemically, electronically and/or by means of radiation to allow for an improved adhesion of paclitaxel and to assist the precipitation or crystallization of the paclitaxel.

By the texturing of the surface of the catheter balloon the surface of the catheter balloon is to be modified in the range from nanometers to micrometers, i.e. a kind of micro-rough surface structure is to be provided. Surface texturing is preferably applied to the whole area to be coated of the catheter balloon and may result in organized or random structures.

The catheter balloons may be composed of the following materials:

parylene C, parylene D, parylene N, parylene F, polyvalerolactones, poly-ε-decalactone, polylactonic acid, polyglycolic acid, polylactides, polyglycolides, copolymers of the polylactides and polyglycolides, poly ε-caprolactone, polyhydroxybutyric acid, polyhydroxybutyrates, polyhydroxyvalerates, polyhydroxybutyrate-co-valerate, poly(1,4-dioxane-2-dione), poly(1,3-dioxane-2-one), poly-para-dioxanone, polyanhydrides, polymaleic acid anhydride, polyhydroxymethacrylates, fibrin, polycyanoacrylate, polycaprolactone dimethylacrylates, poly-β-maleic acid, polycaprolactone butyl acrylates, multiblock polymers from oligocaprolactonedioles and oligodioxanonedioles, polyether ester multiblock polymers from PEG and poly(butylene terephthalate), polypivotolactones, polyglycolic acid trimethyl carbonates, polycaprolactone glycolides, poly(γ-ethyl glutamate) poly(DTH-iminocarbonate), poly(DTE-co-DT-carbonate), poly(bisphenol A-iminocarbonate), polyorthoesters, polyglycolic acid trimethyl-carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyether ester, polyethylene oxide, polyalkene oxalates, polyorthoesters as well as their copolymers, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivatives thereof, heparins, chondroitin sulfate, dextran, β-cyclodextrins, copolymers with PEG and polypropylene glycol, gum arabic, guar, gelatine, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylenes, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones, polysiloxanes, polydimethylsiloxanes, polyvinyl halogens and copolymers, cellulose ethers, cellulose triacetates, chitosans and copolymers and/or mixtures of the aforementioned polymers.

Polyamides, block copolymers of polyamide-polyether-polyester, polyurethanes, polyester and polyolefins are preferred.

It is of importance to avoid all damage to the catheter balloons while the balloon surface is textured and to ensure that their capability to expand is not disadvantageously affected. Thus, the methods for micro texturing the balloon surface must not lead to the formation of holes, micropores or fissures in the balloon material. Ideally, only the outer surface of the balloon, i.e. to a maximum depth of 1 μm, is textured.

The dilatable catheter balloon may be textured mechanically by making use of a rasp-like device, a rasp or a blasting method employing solid particles, such as a sand blasting procedure.

In a chemical-mechanical procedure a suspension or a dispersion of solid particles in a solvent, in particular in water, is used. Such methods are also referred to as chemical polishing methods. By rubbing such compositions onto the surface of the balloon material the material is roughened without deep fissures or holes being created.

In a purely chemical texturing method, acids, bases, etching chemicals and/or oxidizing chemicals corroding the surface of the balloon material are used. Such chemicals, however, are to be used with caution, as the balloon material could be damaged if the exposition period is to long or too intense.

When an electrical or electronic procedure is used for texturing the surface of the dilatable catheter balloon, texturing is performed by means of conductors which are heated by current flow. For example, a fine, warm, hot or glowing needle may be used to melt the surface of the balloon material by means of which certain patterns can be created on the surface, especially when the needle is moved along the surface of the catheter balloon.

An elegant method for generating organized textures, especially in form of micro depressions or micro channels, may include the use of lasers or basically of strongly focused radiation. Said radiation means are very accurate and may be especially used for the generation of defined textures such as grids, spirals or lines.

Such a structuring of the surface results in an extension of the surface and thereby enables a higher loading of the active agent compared to an unstructured surface.

The microstructuring of the catheter balloon thereby creates a three dimensional surface, which can be loaded with a bigger amount of paclitaxil. Furthermore compared to the flat untreated surface of the balloon, the structured surface also enables a better adhesion of the active agent, which then is better protected against being removed by washing and dissolution in the blood stream, as it is at least partly incorporated in the so generated pores.

The textured or micro modified to nano-modified surface of the catheter balloon may be wetted by using all common methods. For example, the solution of paclitaxel in DMSO may be applied onto the balloon surface by means of spattering, dipping, plasma deposition, brushing or spraying. While the whole surface of the catheter balloon is usually coated when a dipping method or plasma disposition are used, spattering, brushing and spraying may be used when only a portion of the balloon surface is to be coated.

According to an embodiment, the catheter balloon does not have to be completely coated. Partial coating of or loading of certain texture elements onto the surface of the catheter balloon may be sufficient. A special catheter balloon including micro-needles or micro-pores or micro-chambers is disclosed in the international patent application no. WO 02/043796 A2 issued to Scimed Life Systems, Inc., USA, wherein inflatable and textured areas are present on the balloon surface. In said embodiment, loading or inflating certain portions of the balloon surface would be sufficient to achieve the desired therapeutic success, wherein it is also possible, evidently, that the whole surface is coated.

Furthermore, another possibility includes coating the catheter balloon partially, i.e. certain sections of the catheter balloon and, successively, additional areas until a completely coated catheter balloon is obtained, if desired.

It has been found that upon complete or partial wetting of the surface of the catheter balloon the desired coating cannot be generated by mere evaporation of the solvent. Thus, step V) of the inventive coating method in which a solvent capable of precipitating paclitaxel is applied onto the wetted surface immediately after the wetting process or immediately after the application of the solution of paclitaxel in DMSO and before the DSMO has evaporated to an extent of 50%, preferably 25% and more preferably 10% is particularly essential. Said additional solvent is added to the DSMO and is intended to reduce the solubility of paclitaxel in DSMO. Said addition solvent is typically designed such that paclitaxel is only poorly soluble or insoluble therein, so that the solubility product of paclitaxel in DSMO is reduced when said solvent is added to the DMSO.

Said additional solvent is used for precipitating paclitaxel and is preferably dispersed homogenously in the DMSO. Precipitating agent is added, preferably by spattering or brushing or pipetting, until a slight clouding is caused or until paclitaxel visibly starts to precipitate. At this point, some more precipitating solvent may be added or the solvent mixture of DMSO and precipitating solvent is left to evaporate. Evidently, the drying process of the coating may be accelerated by applying negative pressure or vacuum; air drying, however, is to be preferred.

The inventive paclitaxel coating which is hard to characterize is thus obtained.

Preferably, such solvents which have a poor solubility of paclitaxel are used as a precipitating solvent for paclitaxel. Generally, the solubility of paclitaxel in such solvents should be characterized by no more than 1 mg of paclitaxel per 1 ml of solvent being soluble.

Water is particularly suited as precipitating solvent, due to the fact that paclitaxel is only very poorly soluble therein and as water is both physiologically acceptable and harmless to the balloon material.

In the best case, distilled, desalted and deionized water which can be obtained by common ion exchange chromatography is used.

It is additionally preferred that the water has a pH in the range of from 2 to 6, preferably from 3 to 5 and more preferably form 3.5 to 4.5. The pH may be adjusted using organic acids and salts of said organic acids, wherein generally as small an amount of buffer in form of acids and conjugated bases as possible is to be used.

Particularly suited acids for adjusting the pH include: formic acid, acetic acid, propionic acid, oxalic acid, salicylic acid, tartaric acid, fumaric acid, gluconic acid, lactic acid, malic acid, ascorbic acid, maleic acid, malonic acid, hydroxymaleic acid, pyruvic acid, phenylacetic acid, benzoic acid, glutaric acid, camphorsulfonic acid or china acid (quinic acid). Additionally, salts of said acids, such as sodium acetate, calcium oxalate or lithium malonate may be used, if required.

Furthermore, another active agent may be added to the solution of paclitaxel in DMSO, which agent may, for example, be selected from the following group:

abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydroxyoxycyclo phosphamide idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, mitoxantrone, mycophenolate mofetil, β-lapachone, podophyllotoxin, podophyllic acid 2-ethylhydrazide, molgramostim (rhuGM-CSF), peginterferon α-2b, lenograstim (r-HuG-CSF), macrogol, selectin (cytokine antagonist), cytokinin inhibitors, COX-2 inhibitor, angiopeptin, monoclonal antibodies inhibiting muscle cell proliferation, bFGF antagonists, probucol, prostaglandins, 1-hydroxy-11-methoxycanthin-6-one, scopoletin, NO donors, pentaerythrityl tetranitrate and sydnoimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof, 6-α-hydroxy-paclitaxel, taxoteres, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF-1, active agents from the group of antibiotics, cefadroxil, cefazolin, cefaclor, cefoxitin, tobramycin, gentamicin, penicillins, dicloxacillin, oxacillin, sulfonamides, metronidazole, enoxaparin, heparin, hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β und γ, histamine antagonists, serotonin blockers, apoptosis inhibitors, apoptosis regulators, halofuginone, nifedipine, tocopherol, tranilast, molsidomine, tea polyphenols, epicatechin gallate, epigallocatechin gallate, leflunomide, etanercept, sulfasalazine, dicloxacillin, tetracycline, triamcinolone, mutamycin, procainimide, retinoic acid, quinidine, disopyrimide, flecamide, propafenone, sotalol, natural and synthetically obtained steroids such as bryophyllin A, inotodiol, maquiroside A, ghalakinoside, mansonine, strebloside, hydrocortisone, betamethasone, dexamethasone, non-steroidal substances (NSAIDS) such as fenoprofen, fenoprofen, ibuprofen, indomethacin, naproxen, phenylbutazone, antiviral agents, acyclovir, ganciclovir zidovudine, clotrimazole, flucytosine, griseofulvin, ketoconazole, miconazole, nystatin, terbinafine, antiprotozoal agents, chloroquine, mefloquine, quinine, natural terpenoids, hippocaesculin, barringtogenol-C21-angelate, 14-dehydroagrostistachin, agroskerin, agrostistachin, 17-hydroxyagrostistachin, ovatodiolids, 4,7-oxycycloanisomelic acid baccharinoids B1, B2, B3 and B7, tubeimoside, bruceantinoside C, yadanziosides N and P, isodeoxyelephantopin, tomenphantopin A and B, coronarin A,B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

Further embodiments relate to catheter balloons and in particular to multifold balloons for catheters coated according to the methods disclosed herein.

The catheter balloons are coated with essentially pure paclitaxel. Thus, the catheter balloons carry a layer that includes an active agent in form of paclitaxel only, wherein in said layer only traces of DMSO and possibly of another solvent and minimum residues of the acid used and possibly of its conjugated base are contained.

Due to the inventive coating method, the paclitaxel dried at the surface of the catheter balloon has a special consistence, which is hard to characterize but seems to be essential for the transfer to the cell wall and the incorporation, especially into the smooth muscle cells.

In the case of multifold balloons, one part of the paclitaxel-containing coating is provided underneath the folds when the balloon is in its compressed state. Said amount is sufficient to achieve the desired therapeutic success even if the remaining uncovered balloon surface is not coated with active agent.

Thus, embodiments also relate to catheters that include a catheter balloon coated according to the methods disclosed herein.

Such catheters are preferably used for treating constricted vessel segments, particularly of blood vessels and for the treatment and prophylaxis of stenosis, restenosis, arteriosclerosis, atherosclerosis and fibrotic vessel constriction.

Furthermore, catheter balloons which are coated according to the methods disclosed herein are suited for the treatment and/or prophylaxis of in-stent restenosis, i.e. a reoccurring vessel constriction within an already implanted stent in such cases where the placement of an additional stent within an already implanted stent would prove to be very problematic or even impracticable from a medical point of view. Such in-stent restenoses can be effectively treated without an additional stent having to be implanted by applying active agent with the help of a catheter coated according to the methods disclosed herein or respectively a catheter balloon of a dilatation catheter which balloon is coated according to the methods disclosed herein.

Furthermore, the catheter balloons coated according to the methods disclosed herein are particularly suited for the treatment of small vessels, preferably such vessels having a vessel diameter of less than 2.25 mm.

The catheter balloons coated according to the methods disclosed herein are preferably used in the cardiovascular area, but the catheter balloons coated according to the methods disclosed herein are also suited for the treatment of vessel constrictions of biliary tracts, esophagus, urinary tracts, pancreas, renal tracts, pulmonary tracts, trachea, small intestine and large intestine.

The following examples illustrate potential embodiments of the methods without limiting the scope of the invention to said precise examples.

### EXAMPLES

### Example 1

A commercially available dilatation catheter with expandable balloon composed of a polyamide is provided.

The surface of the catheter balloon is roughened in the range of nanometers to micrometers by means of sand blasting.

Paclitaxel (commercially available from Sigma, Fermentek, BC Biotech or Arianna International) is dissolved in dried dimethyl sulfoxide (DMSO) at a concentration of 50 mg per ml of DMSO.

The solution of paclitaxel in DMSO is sprayed onto the catheter balloon and subsequently a fine mist of water having a pH of 3.5 is sprayed onto the surface of the catheter balloon which has been wetted with the solution of paclitaxel in DMSO. The water had been distilled beforehand and subsequently the pH was set to 3.5 with acetic acid and sodium acetate. The water mist is sprayed onto the wetted surface until the paclitaxel starts to precipitate.

Subsequently, the surface of the catheter balloon is left to dry in the air and the coating procedure with paclitaxel in DMSO and the subsequent precipitation by means of water mist is repeated twice.

The amount of paclitaxil on the surface of the catheter balloon is 3 μg per mm² balloon surface.

Afterwards, the catheter balloon is dried, sterilized and the whole catheter is packed.

### Example 2

A multifold balloon such as described, for example, in WO 2004/028582 A1, WO 94/23787 A1 or WO 03/059430 A1 is provided. The multifold balloon is provided with a total of 5 folds enclosing a cavity when the balloon is in compressed state and bending outward when it is in expanded state so that the balloon in its expanded state has an essentially tube-like shape.

The multifold balloon is expanded and then its surface is roughened by means of a so called “chemical polishing” process, wherein a suspension of fine particles, preferably in the range of micrometers, is used in said process and said suspension is rubbed onto the surface of the expanded catheter balloon such that a roughened surface is created.

A solution of 80 mg of paclitaxel in 1.0 ml of DMSO and 0.1 ml of methanol is prepared and stirred until the paclitaxel is entirely dissolved.

The roughened expanded catheter balloon is dipped into said solution of paclitaxel in DMSO and methanol and while the balloon rotates a water mist is sprayed upon it, wherein the water has been deionized beforehand by means of an ion exchanger and subsequently it has been adjusted to a pH of 4.0 by means of as small an amount of oxalic acid and calcium oxalate as possible.

Once the first fine paclitaxel crystals have formed, the catheter balloon is left to dry slowly in the air, so that a fine and largely uniform layer of paclitaxel is formed on the catheter balloon. Upon completion of drying the catheter balloon coated for the first time is dipped three more times into the paclitaxel solution and dried afterwards without a water mist being sprayed upon it. The fine paclitaxel crystals on the balloon surface serve as seed crystal for the newly applied paclitaxel. Once the balloon has been dipped into the paclitaxel solution for the fourth time, the methanol and DMSO are left to evaporate and the balloon is then dried under vacuum.

If required, the remaining content of paclitaxel in the DMSO solution may be determined, so that the amount of paclitaxel applied to the catheter balloon can be determined based on the initial concentration.

Generally, from 1 μg to 20 μg of paclitaxel are applied per mm² of coated balloon surface.

After sterilization, the balloon is provided with a protective sheath intended to protect the active agent on the coated dilatable catheter balloon during transport and storage which sheath is removed prior to the insertion of the catheter by the cardiologist.

### Example 3

A commercially available dilatation catheter with expandable balloon made of a polymer is provided.

The catheter balloon includes a block copolymer of polyamide, polyether and polyester or of polyurethane, a polyester or a polyolefin.

Channels or depressions or spirals or grid-like patterns having a depth of from 1 nm to 1 μm are burned into the balloon material by means of a laser.

A solution of 70 mg of paclitaxel in 1.0 ml of DMSO having a water content of about 3 percent by volume is prepared and applied onto the horizontal area of the surface of the catheter balloon by brushing or spattering.

Subsequently, demineralized water adjusted to a pH of 4.8 by a mixture of tartaric acid and sodium tartrate is directly applied onto the surface of the catheter balloon which has been wetted with the solution of paclitaxel by means of a very fine syringe.

Paclitaxel starts to deposit in the textures of the balloon surface. Once the solvent DMSO has evaporated, the catheter balloon is turned and another portion of the balloon surface which is then situated in horizontal position is wetted with the solution of paclitaxel and DMSO and subsequently water is applied onto said wetted surface until the paclitaxel precipitates, as described before. The wetting and precipitation process is repeated until the complete balloon surface is coated.

Subsequently, the catheter balloon is thoroughly dried and an amorphous paclitaxel coating is obtained which is predominantly contained in the micro textures.

The amount of paclitaxel on the catheter balloon is 2-4 μg per mm² balloon surface.

After sterilization, the balloon is provided with a protective sheath intended to protect the active agent on the coated dilatable catheter balloon during transport and storage which sheath is removed prior to the insertion of the catheter by the cardiologist.

### Example 4

On top of the catheter balloon corresponding to Example 1, an uncoated cobalt-chrom-stent is attached.

### Example 5

On top of the catheter balloon corresponding to Example 1, a paclitaxel coated cobalt-chrom-stent is attached.

### Example 6

General instructions regarding the execution of the dilatation using the coated catheter balloon.

The catheter balloon is coated with 3 μg compact paclitaxel per mm² balloon surface. An average patient is pretreated with approximately 325 mg acetylsalicylic acid and approximately 300 mg clopidogrel 12 and 2 hours before dilatation. Prior to the engagement Heparin is administered intravenously in an amount that the coagulation time is more than 250 seconds. Subsequently an inhibitor of glycoprotein Ib/IIIa is administered in an amount that is determined by the physician and a guide wire is fed forward to the target region. Afterwards the catheter balloon, which is coated according to the methods disclosed herein is implemented and the target region is dilated with 6 to 8 atm (approximately 6000 to 8000 hPa) for about one minute and if necessary deflated and another one, two or three times inflated with approximately 8000 hPa.

### Example 7

A 66 year old patient, who is a former smoker and who is suffering from hypercholesteremia is further suffering from angina, which not is accompanied by shortness of breath and dizziness.

After severe ischemia in peripheral veins was documented using the Phase III Bruce Stress Test, an angiography diagnosis was performed.

The coronary angiography resulted in the finding of an occlusion of the RCA with collateralization of the left coronary system. Subsequently a recanalization of the RCA with an uncoated metal stent was performed.

After about a year the patient was still asymptomatic and a Thallium-201 liability/stress test was performed, which showed a hypoperfusion in the lower vessel wall.

Two month later a coronary angiography was performed, which indicated a diffuse in stent restenosis (ISR) of the proximal, middle and distal part of the RCA and a collateralisation of the posterior sloping coronary artery (PDA) with a 70% stenosis of the middle tract.

A further PTCA using a cutting balloon was performed at the patient over the entire length of the RCA followed by a brachytherapy in the proximal, middle and distal area of the RCA and PDA coronary artery by implementing an uncoated metal stent in the middle area of the LCX.

After about six month a control angiography was performed which acknowledged a good intermediate result with regard to the PCTA in the RCA and PDA coronary arteries but which was associated with an occlusion of the PL artery, which could not be reopened despite several attempts using different guide wires.

After about three years the patient subjected himself again to a stress test and the cardiogram displayed two abnormalities. Thereupon a further coronary angiography was performed, which showed a 70% ISR in the proximal area and a 95% ISR in the distal areas of the RCA as well as a 60% stenosis of the PDA and PL coronary artery.

The proximal and distal lesions of the RCA were treated with a cipher stent implementation.

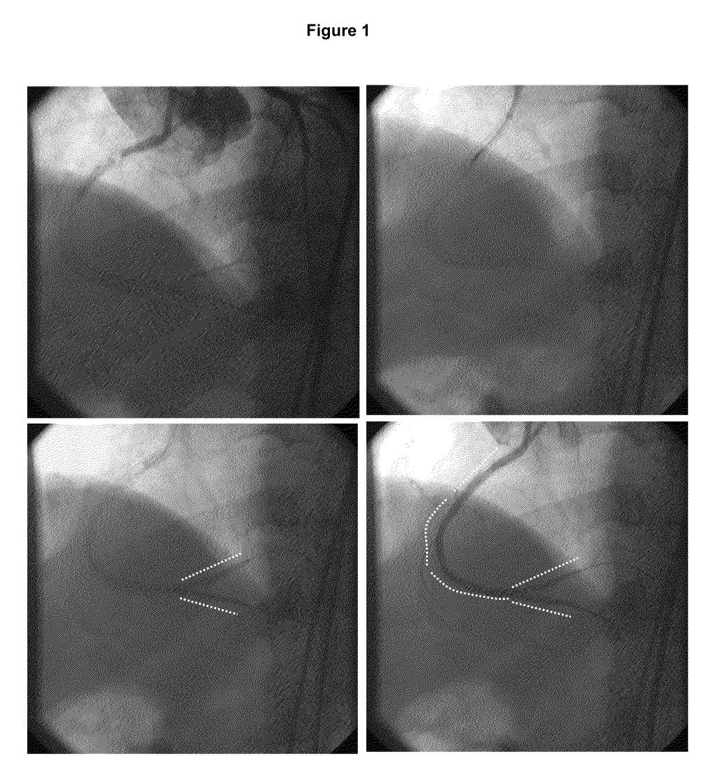

After 2.5 more years a further stress test was performed, which again displayed abnormalities in the inferior and posterio lateral areas. A coronary angiography this time resulted in a complete occlusion of the RCA with collateralisation from the left till the right coronary system (FIG. 1, picture A) together with a strong atheromasie of the PDA and PL coronary artery.

By introducing a Judkins right 8Fr guiding catheter and a Judkins diagnosis catheter the ISR occlusion was penetrated with a miracle 3 guide wire, which was directed until it reached the PDA coronary artery. After predilation with a avion 1.5×14 mm balloon, two passages were generated by using a directional coronary atherectomy catheter 2.6 mm (FoxHollow, Silver Hawks, Calif./FIG. 1, picture B), to remove part of the neointimal tissue. Subsequently the guide wire was introduced into the PL coronary artery.

Despite the presence of several stents, the introduction of a 2.5×13 paclitaxel eluting catheter balloon (DIOR, Eurocor, Germany) succeeded with the help of the kissing balloon procedure. It further succeeded to dilate the catheter balloon with 14 atm (14186 hPa) for two minutes (FIG. 1, picture C). Subsequently two Taxus 2.25×24 mm stents were placed concurrently in the proximal area of the PL and PDA coronary arteries and in the distal area of the RCA and the arteries were again dilated with paclitaxel eluting balloon for two minutes with a pressure of 22 atm (22292 hPa). Afterwards a Taxus 4.0×32 mm stent was implanted in the middle and proximal area of the RCA, which resulted in a very good and immediate angiography result (FIG. 1, picture D). Within the subsequent 12 month the patient remained asymptomatic.

### Example 8

A 53 year old patient, who is a smoker, is suffering from NIDDM and dyslipidemia and is prestressed with a family history of ischemic heart diseases.

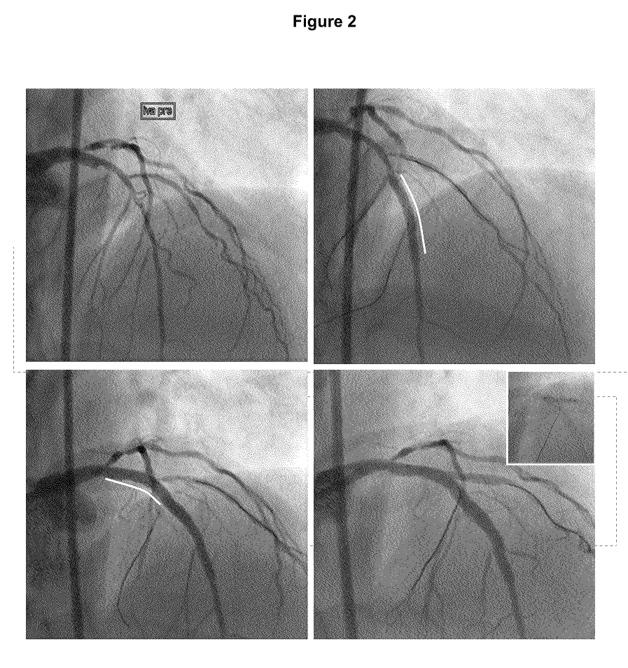

1993 the patient was suffering from an acute myocardial infarction and thereupon was subjected to a primary PCI of the LAD and LCX coronary artery without stent implantation. Coronary angiography displayed an angiographically normal left aorta and a bifurcation lesion, which included the LAD/D1, with a 90% stenosis of the LAD, a 80% stenosis of the first diagonal (FIG. 2, picture A), furthermore a 90% stenosis of the LCX in the proximal area and a 90% stenosis in a dominant RCA in the middle area.

After a meeting with the patient a complete revascularization strategy was chosen. After a guide wire was placed within the LAD, the first diagonal and the second septal branching for protection, a IVUS was performed to examine the bifurcation lesion and to choose the correct size for the balloon and the stent. The IVUS affirmed the result for the LAD as well as for the D1 and displayed a diffuse indisposition of the LAD.

To avoid the double placing of a stent in the bifurcation of a diabetes patient, a provisional stenting technique for the branch was chosen.

A Quantum 3.0×17 (Boston scientific) was chosen for a predilation at 20 atm (20265 hPa), followed by a 3.0×28 mm Xience stent (Abbot Vascular) implantation (FIG. 2, picture B). Afterwards the diagonal branch was dilated by using a DIOR paclitaxel-eluting balloon (Eurocor GmBH, Germany) 3.0×17 at 10 atm (10133 hPa) for 2.0 minutes. Next a 3.5×28 mm Xience Stent was implanted into the bifurcation lesion, followed by a final “kissing balloon” inflation (Quantum 3.5×13 of the LAD at 8 atm (8106 hPA)) and DIOR paclitaxel-eluting balloon 3.0×17 of the D1 at 10 atm. (10133 hPa)). An excellent angiographic result was obtained (FIG. 2, picture C and D).

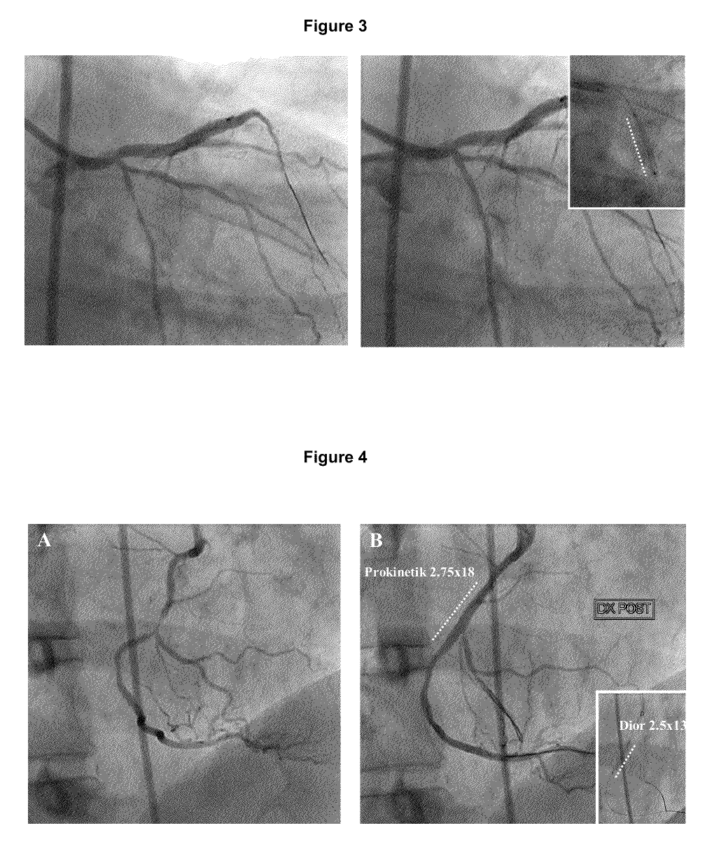

Subsequently a stent like result was obtained by dilatation of the proximal segment of the LCX using a multidilation strategy and a 2.5×13 mm DIOR paclitaxel-eluting balloon at 16 atm and extended inflation (total time of inflation: 4.5 minutes, FIG. 3 picture A and B).

Concluding, a guide wire was introduced into the lesion of the RCA coronary artery after protection of the acute marginal branches and a predilation was performed by using a 2.5×13 mm DIOR paclitaxel-eluting balloon at 14 atm (14186 hPa) for 1.45 minutes. Then an uncoated cobalt-chrom-stent with thin branches (Prokinetic 2.75×18 at 8 atm (8106 hPa), Biotronic) was implanted and an immediate excellent angiography result was obtained (FIG. 4, picture A and B).

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. Method for loading dilatable catheter balloons comprising:
I) providing a dilatable catheter balloon;
II) providing a solution of paclitaxel in dimethyl sulfoxide;
III) texturing the surface of the dilatable catheter balloon;
IV) wetting the surface of the dilatable catheter balloon with the solution of paclitaxel in dimethyl sulfoxide;
V) applying a solvent which is capable of precipitating paclitaxel onto the surface of the dilatable catheter balloon wetted with the solution of paclitaxel in dimethyl sulfoxide;
VI) drying the wetted surface of the dilatable catheter balloon;

2. Method according to claim 1, wherein the dilatable catheter balloon is a multifold balloon.

3. Method according to claim 1, wherein the dilatable catheter balloon is coated in its expanded state.

4. Method according to claim 2, wherein the dilatable multifold balloon is coated in its expanded state.

5. Method according to claim 1 further comprising step VII):
VII) Refolding of the multifold balloon into its compressed state.

6. Method according to claim 1, wherein the solution of paclitaxel in dimethyl sulfoxide contains from 1 mg to 150 mg of paclitaxel per 1 ml of dimethyl sulfoxide.

7. Method according to claim 6, wherein the solution of paclitaxel in dimethyl sulfoxide contains from 40 mg to 60 mg of paclitaxel per 1 ml of dimethyl sulfoxide.

8. Method according to claim 1, wherein the dimethyl sulfoxide used has a water content of less than 5 percent by volume.

9. Method according to claim 1, wherein the dimethyl oxide used may contain an additional solvent in an amount of up to 10 percent by volume.

10. Method according to claim 1, wherein at least one carrier substance is added to the solution of paclitaxel in dimethyl sulfoxide.

11. Method according to claim 10, wherein the at least one carrier substance is selected from the group consisting of:
parylene C, parylene D, parylene N, parylene F, polyvalerolactones, poly-ε-decalactone, polylactonic acid, polyglycolic acid, polylactides, polyglycolides, copolymers of the polylactides and polyglycolides, poly ε-caprolactone, polyhydroxybutyric acid, polyhydroxybutyrates, polyhydroxyvalerates, polyhydroxybutyrate-co-valerate, poly(1,4-dioxane-2,3-dione), poly(1,3-dioxane-2-one), poly-para-dioxanone, polyanhydrides, polymaleic acid anhydride, polyhydroxymethacrylates, fibrin, polycyanoacrylate, polycaprolactone dimethylacrylates, poly-β-maleic acid, polycaprolactone butyl acrylates, multiblock polymers from oligocaprolactonedioles and oligodioxanonedioles, polyether ester multiblock polymers from PEG and poly(butylene terephthalate), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polyglycolic acid trimethyl-carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide soft polyurethanes, polyurethanes having amino acid residues in the backbone, polyether esters, polyethylene oxide, polyalkene oxalates, polyorthoesters as well as their copolymers, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivatives thereof, heparins, chondroitin sulfate, dextran, β-cyclodextrins, copolymers with PEG and polypropylene glycol, gum arabic, guar, gelatin, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylene, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones, polysiloxanes, polydimethylsiloxanes, polyvinyl halogens and copolymers, cellulose ethers, cellulose triacetates, chitosans and copolymers and/or mixtures of the aforementioned polymers.

12. Method according to claim 1, wherein the surface of the dilatable catheter balloon is textured mechanically, chemically, electronically and/or by means of radiation.

13. Method according to claim 12, wherein the mechanical texturing of the surface of the dilatable catheter balloon is achieved by means of a sandblasting process or by using a rasp.

14. Method according to claim 12, wherein the chemical texturing of the surface of the dilatable catheter balloon is achieved by means of acidic, basic, etching or oxidizing chemicals.

15. Method according to claim 12, wherein the electrical texturing of the surface of the dilatable catheter balloon is achieved by means of conductors heated by current flow.

16. Method according to claim 12, wherein the texturing of the surface of the dilatable catheter balloon is achieved by means of laser radiation or by means of strongly focusable radiation.

17. Method according to claim 1, wherein the wetting of the surface of the dilatable catheter balloon is completely or partially achieved by means of spraying, dipping, plasma deposition, brushing or spattering.

18. Method according to claim 1, wherein steps IV) to VI) are repeated several times.

19. Method according to claim 1, wherein the solvent capable of precipitating paclitaxel has a solubility of paclitaxel in said solvent of less than 1 mg of paclitaxel per 1 ml of solvent.

20. Method according to claim 1, wherein the solvent capable of precipitating paclitaxel is water.

21. Method according to claim 20, wherein the water is distilled, desalted or deionized water.

22. Method according to claim 20, wherein the water has a pH of from 3 to 5.

23. Method according to claim 22, wherein the pH of the water is adjusted by means of formic acid, acetic acid, propionic acid, oxalic acid, salicylic acid, tartaric acid, fumaric acid, gluconic acid, lactic acid, malic acid, ascorbic acid maleic acid, malonic acid, hydroxymaleic acid, pyruvic acid, phenylacetic acid, benzoic acid, glutaric acid, camphorsulfonic acid or china acid (quinic acid).

24. Method according to claim 1, wherein the solvent capable of precipitating paclitaxel is added by means of spray injection, spraying or pipetting.

25. Method according to claim 1, wherein the drying process of the wetted surface of the dilatable catheter balloon is accelerated by a vacuum.

26. Method according to claim 1, wherein the dilatable catheter balloon includes a material or a mixture of materials, wherein the material or the mixture of materials is selected from the following group of materials:
parylene C, parylene D, parylene N, parylene F, polyvalerolactones, poly-ε-decalactone, polylactonic acid, polyglycolic acid, polylactides, polyglycolides, copolymers of the polylactides and polyglycolides, poly-ε-caprolactone, polyhydroxybutyric acid, polyhydroxybutyrates, polyhydroxyvalerates, polyhydroxybutyrate-co-valerate, poly(1,4-dioxane-2,3-dione), poly(1,3-dioxane-2-one), poly-para-dioxanone, polyanhydrides, polymaleic acid anhydride, polyhydroxymethacrylates, fibrin, polycyanoacrylate, polycaprolactone dimethylacrylates, poly-β-maleic acid, polycaprolactone butyl acrylates, multiblock polymers from oligocaprolactonedioles and oligodioxanonedioles, polyether ester multiblock polymers from PEG and poly(butylene terephthalate), polypivotolactones, polyglycolic acid trimethyl carbonates, polycaprolactone glycolides, poly(γ-ethyl glutamate) poly(DTH-iminocarbonate), poly(DTE-co-DT-carbonate), poly(bisphenol A-iminocarbonate), polyorthoesters, polyglycolic acid trimethyl-carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyether ester, polyethylene oxide, polyalkene oxalates, polyorthoesters as well as their copolymers, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivatives thereof, heparins, chondroitin sulfate, dextran, β-cyclodextrins, copolymers with PEG and polypropylene glycol, gum arabic, guar, gelatine, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylene, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones, polysiloxanes, polydimethylsiloxanes, polyvinyl halogens and copolymers, cellulose ethers, cellulose triacetates, chitosans and copolymers and/or mixtures of the aforementioned polymers.

27. Method according to claim 1, wherein an additional active agent is incorporated into the solution of paclitaxel in dimethyl sulfoxide, selected from the group consisting of:
abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydroxyoxycyclo phosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, mycophenolate mofetil, β-lapachone, podophyllotoxin, podophyllic acid-2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, angiopeptin, monoclonal antibodies inhibiting muscle cell proliferation, bFGF antagonists, probucol, prostaglandins, 1-hydroxy-11-methoxycanthin-6-one, scopoletin, NO donors, pentaerythrityl tetranitrate and sydnoimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof, 6-α-hydroxy-paclitaxel, taxoteres, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF-1, active agents from the group of antibiotics, cefadroxil, cefazolin, cefaclor, cefoxitin, tobramycin, gentamicin, penicillins, dicloxacillin, oxacillin, sulfonamides, metronidazole, enoxaparin, heparin, hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, halofuginone, nifedipine, tocopherol, tranilast, molsidomine, tea polyphenols, epicatechin gallate, epigallocatechin gallate, leflunomide, etanercept, sulfasalazine, tetracycline, triamcinolone, mutamycin, procainimide, retinoic acid, quinidine, disopyrimide, flecamide, propafenone, sotalol, natural and synthetically obtained steroids such as bryophyllin A, inotodiol, maquiroside A, ghalakinoside, mansonine, strebloside, hydrocortisone, betamethasone, dexamethasone, non-steroidal substances (NSAIDS), fenoprofen, ibuprofen, indomethacin, naproxen, phenylbutazone, antiviral agents, acyclovir, ganciclovir zidovudine, clotrimazole, flucytosine, griseofulvin, ketoconazole, miconazole, nystatin, terbinafine, antiprotozoal agents, chloroquine, mefloquine, quinine, natural terpenoids, hippocaesculin, barringtogenol-C21-angelate, 14-dehydroagrostistachin, agroskerin, agrostistachin, 17-hydroxyagrostistachin, ovatodiolids, 4,7-oxycycloanisomelic acid baccharinoids B1, B2, B3 and B7, tubeimoside, bruceantinoside C, yadanziosides N and P, isodeoxyelephantopin, tomenphantopin A and B, coronarin A,B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

28. Coated dilatable catheter balloon which can be obtained by the method according to claim 1.

29. Dilatation catheter comprising the coated dilatable catheter balloon according to claim 28.

30. Dilatation catheter according to claim 29 further comprising a coated or uncoated stent attached to the coated dilatable catheter balloon.
